(19) United States
(12) Patent Application Publication
(10) Pub. No.: US 2025/0195817 A1
(43) Pub. Date: Jun. 19, 2025

(54) ELBOW ASSEMBLY FOR DIRECT CONNECTION TO A FACE MASK

(71) Applicant: DCSTAR INC, New York, NY (US)

(72) Inventor: David LUO, New York, NY (US)

(73) Assignee: DCSTAR INC, New York, NY (US)

(21) Appl. No.: 18/540,118

(22) Filed: Dec. 14, 2023

(51) Int. Cl. A61M 16/08; A61M 16/06

(52) U.S. Cl. CPC A61M 16/0816; A61M 16/06; A61M 16/0616; A61M 16/0633; A61M 16/209

(57) ABSTRACT

A ventilation elbow assembly, which is directly connectable to a face mask. The elbow assembly includes an elbow, connector, ventilation port, noise reduction device, and quick-release connector. The elbow has a first interface, second interface, and annular channel. At the first interface, there is at least one stepped portion, which is configured to connect to the face mask and seal pressurized gas. A baffle is provided at the first interface, limiting frame movement relative to the face mask and elbow. The assembly also includes an anti-asphyxiation valve to prevent the patient from asphyxiation without continuous positive pressure in the elbow. With a connector, the elbow can first connect to the connector before the face mask, expanding the range of movement of the elbow assembly relative to the face mask. This is configured to increase the range of movement of the elbow assembly relative to the face mask.

### TECHNICAL FIELD

The present disclosure relates to a ventilation elbow assembly directly connectable to a face mask. It primarily involves an elbow, a connector, a ventilation port provided on the elbow or connector, a noise reduction device, and a quick-release connector. These components work in conjunction with a tube, a face mask, and a frame to deliver pressurized air from a ventilator to a patient's airway for therapeutic purposes.

### BACKGROUND

Obstructive Sleep Apnea (OSA) is a disease that is closely associated with a patient's respiratory system and sleep quality. It poses significant risks to various organs and the overall well-being of a person. Today, there are various solutions and technologies from different fields used for diagnosing, monitoring, and treating patients with sleep apnea. Continuous Positive Airway Pressure (CPAP) therapy is a commonly employed treatment method at present. This therapy involves delivering a continuous positive pressure of air from a respiratory machine to a patient's airway. By increasing air pressure and reducing airway resistance, it helps keep the upper airway unobstructed, thereby reducing the occurrence of breathing pauses. The treatment of obstructive sleep apnea involves the coordinated use of various treatment components. The interaction between these components impacts treatment effectiveness, comfort, and patient compliance. Therefore, in addition to a better understanding of the treatment process and principles, improving the coordination between treatment components and enhancing their comfort during the treatment process is also a way to ensure the best treatment outcomes.

The ventilation elbow is one of the most crucial components in CPAP therapy and is typically located at the exit point of the airflow through the breathing tube. It serves to direct the continuous positive air pressure flow to the patient interface, such as a face mask, nasal face mask, or nasal cushion. Some elbows not only guide the continuous positive air pressure but also facilitate the expulsion of the patient's exhaled air to prevent the re-breathing of carbon dioxide. When compared to CPAP treatment setups for sleep apnea without an elbow, the positive air pressure output from the CPAP machine can sometimes be too forceful. The presence of the elbow helps reduce the direct impact of the airflow on the face mask or the patient's airway, making the airflow more even and thereby reducing patient discomfort. Elbows can also reduce noise and improve the airflow direction. However, they increase air resistance to some extent and may slightly decrease the overall efficiency of the airflow in the meantime. While this impact is typically minimal for patients, in some cases, it might affect the pressure settings on the CPAP machine. Moreover, in setups where the elbow is connected to the face mask via a frame seen in the market, there can be instances of loosening during use due to multiple connection points. When this occurs, patients need to inspect and adjust the elbow and the entire respiratory accessory combination, which can be quite inconvenient for them.

Most of the elbows available on the market share a similar design, with the primary and essential feature being a connection with a first end and a second end. Over time, elbows have incorporated additional functionalities such as noise reduction through materials or direct integration with noise-reducing mesh, and the ability to connect to external parts. Particularly, the first end of the elbow is configured to connect to a frame, while the second end connects to the breathing tube. The connection between the two ends of the elbow and the frame or breathing tube must ensure that there is no gas leakage of the continuous positive air pressure flows at the connection points.

The accuracy and stability of the seal at the connection points remain a challenge to be addressed. Additionally, given the prevalence of elderly individuals among sleep apnea patients, ease of connection at both ends of the elbow with the corresponding components is an aspect that needs optimization. A convenient connection is more advantageous for patients in terms of wearing and adjusting the treatment accessories, making the overall operation simpler for them.

In the CPAP treatment process, aside from the elbow, the frame is also a critical component. Its primary function is to support and secure the face mask, allowing it to fit snugly on the patient's face while ensuring the smooth delivery of airflow and treatment effectiveness. In the market for sleep apnea medical components, several manufacturers offer various types of frames to meet different needs of patients. However, the compatibility of different frames with face masks may vary. The market demands frames that are as lightweight as possible while still securely holding the face mask in place. Furthermore, these frames should be adaptable to the varying needs of different patients, aiming to provide enhanced comfort and effectiveness.

In summary, the current designs of elbows on the market have various areas that can be improved. The air-tightness of these elbows and their compatibility with frames and face masks need optimization. With an increasing variety of face masks available on the market, there are now more diverse options for frames used to secure these face masks. However, the existing frames not only involve connections and compatibility with face masks but also with elbows. Therefore, when patients are selecting and purchasing these components, both the first and second ends of the connector for the face mask must match the connection points of the patient's existing elbow and face mask. While it may seem that the increasing variety of face masks offers more choices for patients, it actually complicates the patient's decision-making process.

### SUMMARY

As a solution, an elbow that directly connects to the face mask is provided in the disclosure, which simplifies the coordination between the frame, elbow, and face mask. This design also enhances the stability of the seal at the connection point of the elbow during the treatment process. Compared to the scenario where the elbow connects to the frame, which then connects to the face mask, this approach simplifies the connection steps and reduces the risk of loosening and air leakage. In addition to ensuring proper sealing, this design increases its functionality by adding features such as noise reduction and quick-release functions. Overall, this design improves some of the drawbacks in the coordination of components of the elbow and has a positive impact on patients.

The goal of this disclosure is to provide a new type of elbow assembly that offers improved sealing and stability. It is configured to be simple to manufacture with low research and production costs, overcoming the limitations present in similar products within the existing technology. This provides a more effective and widely applicable combination of usage scenarios than existing technologies, offering a more simple approach and bringing a better wearing experience to users.

In one embodiment, an elbow assembly for direct connection to a face mask is provided. The elbow assembly is configured to deliver continuous positive pressure gas to a patient's airway to ameliorate sleep-associated breathing disorders. The elbow assembly includes an elbow, which has a first interface for direct connection to the face mask, a second interface for receiving pressurized gas, and an annular channel with a shaft bend between the first interface and the second interface. There is a first interface end on the elbow that includes at least one stepped portion, which is configured to come into close contact with an inner wall of an extended interface at a central opening of the face mask when the stepped portion and the face mask are connected, preventing the elbow from easily detaching when a force is applied to the elbow in a direction opposite to the patient's face, and enabling mutual rotation between the elbow and the face mask. The elbow has a baffle near the first interface, which is configured to ensure that upon a connection of the elbow with the face mask, there exists a gap between an inner surface of the baffle facing the patient's face and an outer surface of the central opening on the face mask, with the gap being configured to at least accommodate a wall thickness of the frame and to limit forward and backward movement of the frame relative to the face mask and the elbow. The elbow assembly further includes an anti-asphyxiation valve, which includes an anti-asphyxiation valve opening and a silicone piece positioned at a lower end of the anti-asphyxiation valve opening with the anti-asphyxiation valve opening set on a wall of the annular channel on the elbow. The anti-asphyxiation valve opening is configured to allow breathing via the anti-asphyxiation valve opening in an absence of continuous positive pressure in the elbow, preventing the patient from asphyxiation. A distance between the inner surface of the baffle at the first interface end of the elbow facing the patient's face, and the first interface of the elbow, is no less than 2 mm.

In an embodiment, an edge of the first interface is a discontinuous circular ring, and the ring has at least one notch to facilitate the snap-fitting of the first interface end with the face mask.

In an embodiment, the baffle is a radially protruding part on an outer wall of the elbow or is formed by the stepped portion on the elbow.

In an embodiment, the anti-asphyxiation valve opening forms a symmetry with the silicone piece at an axis of a second interface end of the elbow.

In an embodiment, the anti-asphyxiation valve opening is configured to have one or more openings.

In another embodiment, an elbow assembly for direct connection to a face mask is provided. The elbow assembly is configured to deliver continuous positive pressure gas to a patient's airway to ameliorate sleep-associated breathing disorders. The assembly includes an elbow, which has a first interface for direct connection to the face mask, a second interface for receiving pressurized gas, and an annular channel with at least one shaft bend between the first interface and the second interface. A partially spherical-shaped protrusion is provided at a first interface end of the elbow, and an outer wall near the first interface end has two protrusions facing outward or two grooves facing inward. The protrusions or grooves adapt with a receiver on an inner wall of a central opening on the face mask to form a hinged connection to allow the elbow to be rotated relative to the face mask along an axis of a formed hinge, and the partially spherical-shaped protrusion at the first interface end is configured to ensure smooth rotation of the elbow and prevent any air leakage during rotation of the elbow. The elbow assembly further includes an anti-asphyxiation valve which includes an anti-asphyxiation valve opening and a silicone piece positioned at a lower end of the anti-asphyxiation valve opening, in which the anti-asphyxiation valve opening is set on a wall of the annular channel on the elbow and is configured to allow breathing via the anti-asphyxiation valve opening in an absence of continuous positive pressure in the elbow, preventing the patient from asphyxiation.

In an embodiment, the elbow has a baffle near the first interface, which is configured to ensure that upon a connection of the elbow to the face mask, there exists a gap between an inner surface of the baffle facing the patient's face and an outer surface of the central opening on the face mask, for accommodating a frame and limiting a back and forth movement of the frame relative to the face mask and the elbow, and a minimum width of the gap is 0.5 mm, corresponding to a wall thickness of the frame at a connection point.

In an embodiment, there is an extended interface at the central opening of the face mask and a distance from the first interface of the elbow to an inner surface of the baffle facing the patient's face is greater than a distance from an outer surface of the central opening on the face mask to a furthest end of the extended interface.

In an embodiment, the anti-asphyxiation valve opening forms a symmetry with the silicone piece at an axis of a second interface end of the elbow.

In yet another embodiment, an elbow assembly for direct connection to a face mask is provided. The elbow assembly is configured to deliver continuous positive pressure gas to a patient's airway to ameliorate sleep-associated breathing disorders. The assembly includes an elbow, which has a first interface for direct connection to the face mask, a second interface for receiving pressurized gas, and an annular channel with a shaft bend between the first interface and the second interface; and a connector, which is provided with a first end connectable to the elbow and a second end directly connectable to the face mask. The second end has at least one stepped portion configured to be in close contact with an extended interface at a central opening of the face mask when the stepped portion and the face mask are connected, preventing the connector from easily detaching when a force is applied to the connector in a direction opposite to the patient's face. The elbow assembly further includes an anti-asphyxiation valve, which includes an anti-asphyxiation valve opening and a silicone piece positioned at a lower end of the anti-asphyxiation valve opening with the anti-asphyxiation valve opening set on a wall of the annular channel on the elbow. The anti-asphyxiation valve opening is configured to allow breathing via the anti-asphyxiation valve opening in an absence of continuous positive pressure in the elbow, preventing the patient from asphyxiation, and the connector has a baffle with a distance between an inner surface of the baffle facing the patient's face and the first interface of the elbow being at least 2 mm.

In an embodiment, the first end of the connector connectable to the elbow is configured to connect to the elbow through a snap-fitting, ball-and-socket joint, or a combination of hinge and ball-and-socket joint.

In an embodiment, the connector is not integrally molded with the elbow and the elbow assembly further includes a ventilation port, which is configured to allow the patient to exhaust waste gases into an external environment.

In an embodiment, given a same elastic modulus of a material, a ratio of a uniform wall thickness at the stepped portion near the first interface of the elbow to an area ratio between a contact area where the first interface of the elbow and the extended interface of the face mask make contact, relative to an annular area formed by their contact, is not equal to the value of the uniform wall thickness of the extended interface of the face mask.

In an embodiment, an area of a side of the silicone piece close to the anti-asphyxiation valve is greater than a total area of the anti-asphyxiation valve opening to prevent the silicone piece from being blown out.

In another embodiment, an elbow assembly for direct connection to a face mask is provided. The elbow assembly is configured to deliver continuous positive pressure gas to a patient's airway to ameliorate sleep-associated breathing disorders. The assembly includes an elbow, which has a first interface for direct connection to the face mask, a second interface for receiving pressurized gas, and an annular channel with a shaft bend between the first interface and the second interface; a connector, provided with a first end connectable to the elbow and a second end directly connectable to the face mask, the second end having at least one stepped portion configured to be in close contact with an extended interface at a central opening on the face mask when the stepped portion and the face mask are connected, preventing the connector from easily detaching when a force is applied to the connector in a direction opposite to the patient's face; and a ventilation port, with at least one through hole, configured to allow the patient to exhale exhaust gases to an external environment. The connector is provided with a baffle configured to leave a gap that can at least accommodate a frame between an inner surface of the baffle facing the patient's face and an outer surface of a central opening on the face mask when the connector is connected to the face mask to limit forward and backward movement of the frame relative to the face mask and the elbow. The elbow assembly further includes an anti-asphyxiation valve that includes an anti-asphyxiation valve opening and a silicone piece at a lower end of the anti-asphyxiation valve opening. The anti-asphyxiation valve opening is provided on a wall of the annular channel on the elbow to allow breathing via the anti-asphyxiation valve opening in an absence of continuous positive pressure in the elbow, preventing the patient from asphyxiation.

In an embodiment, a thickness between the outer surface of the central opening on the face mask and an inner surface of the baffle facing the patient's face is greater than or equal to a wall thickness of the frame at a connection point when the elbow is connected to the face mask.

In an embodiment, the ventilation port includes a noise-reducing material, and the noise-reducing material includes noise-reducing cotton or noise-reducing mesh, the noise-reducing material having one or more of the following characteristics:
A. a thickness of at most 9 mm;
B. a weight of at most 7 g.

In an embodiment, the noise-reducing material covers the ventilation port, and the elbow is provided with at least one port cover for connecting to an outer wall of the annular channel in contact with air to secure the noise-reducing material.

In yet another embodiment, an elbow assembly for direct connection to a face mask is provided. The elbow assembly is configured to deliver continuous positive pressure gas to a patient's airway to ameliorate sleep-associated breathing disorders. The assembly includes an elbow, which has a first interface for direct connection to the face mask, a second interface for receiving pressurized gas, and an annular channel with a bend between the first interface and the second interface. A first interface end on the elbow is configured to be in close contact with an extended interface at a central opening of the face mask. The elbow has a baffle near the first interface, which is configured to ensure that upon a connection of the elbow to the face mask, there exists a gap between an inner surface of the baffle facing the patient's face and an outer surface of the central opening on the face mask, with the gap being configured to at least accommodate a wall thickness of the frame and to limit forward and backward movement of the frame relative to the face mask and the elbow. The elbow assembly further includes a quick-release connector, which has a first end and a second end, the first end configured to be detachably connectable to the second interface end of the elbow, and the second end is configured to be connectable to a breathing tube; and an anti-asphyxiation valve that includes an anti-asphyxiation valve opening and a silicone piece at a lower end of the anti-asphyxiation valve opening. The anti-asphyxiation valve opening is configured to be set on a wall of the annular channel on the elbow to allow breathing via the anti-asphyxiation valve opening in an absence of continuous positive pressure in the elbow, preventing the patient from asphyxiation.

In an embodiment, the second interface of the elbow is connectable to the quick-release connector, and the quick-release connector is configured to have connection portions integrally molded with its main body on both sides for clamping and snapping together, the quick-release connector being rotatable relative to the elbow after a connection to the elbow.

In an embodiment, a thickness between the outer surface of the central opening on the face mask and the inner surface of the baffle facing the patient's face is greater than or equal to a wall thickness of the frame at a connection point when the elbow is connected to the face mask.

In an embodiment, an edge of the first interface is a discontinuous circular ring with at least one notch to facilitate a snap-fitting of the first interface end with the face mask.

In an embodiment, the first interface end on the elbow includes at least one stepped portion, and the baffle can be a wall of the elbow formed relative to the stepped portion at the first interface. The baffle has at least one of the following characteristics:
A. Being perpendicular to an axis of an interface connectable to the face mask;
B. When connected, a distance from the baffle to the outer surface of the central opening on the face mask being at least equal to the wall thickness of the frame at the connection point;
C. Having a height of at least 2 mm;
D. Having a thickness at or between 0.5 to 8 mm.

Implementing the elbow assembly for direct connection to a face mask of this disclosure has at least the following beneficial effects:
1. By having the elbow directly connect with the face mask without a frame, it reduces the number of seams at the connection points between components. The continuous positive pressure airflow from the ventilator passes through the elbow and the patient interface via a breathing tube, and enters the patient's airway. During this process, there is a potential for airflow to leak from the seams. Reducing the number of seams decreases the risk of airflow leakage, thereby improving the overall sealing of the respiratory treatment component system. Traditional installation methods required alignment at two separate locations, first aligning the frame with the fixed face mask and then aligning the elbow with the fixed frame. This disclosure simplifies the installation process from three steps to two. It is only necessary to place the frame onto the elbow before connecting the elbow to the face mask. This optimization of the installation process not only reduces the error rate and saves time but also enhances the user experience, making it easier for people with limited dexterity to operate.
2. In this disclosure, during the installation and disassembly process, the frame only passes through the first interface of the elbow. The frame is secured to the face mask and the elbow through the shape of the face mask and the baffle of the elbow. Therefore, the most critical and indispensable structure of the frame at the connection is the frame opening that passes through the first interface end of the elbow. This disclosure simplifies the connection of the frame to a smooth-walled frame opening, modularizing the frame. For different patient needs, as long as the frames have the same-sized opening that can pass through the first interface end of the elbow, they can be securely connected to the elbow and face mask. This eliminates the need for designing frames with different connecting ports to match various face masks and elbow interfaces. Similarly, there's no need to design face masks and elbows with different connecting ports to fit the frames. The reduction in processing steps for connecting ports significantly reduces the complexity and R&D costs for the elbow, face mask, and frame. It also simplifies the market choices for frames. Additionally, the reduction in structure and processing steps leads to less wastage of raw materials. This is a crucial step towards carbon neutrality, further aligning with the goals of alleviating the environmental and climate change burden, promoting sustainable development, and achieving carbon neutrality.
3. Compared to the connection style where the frame connects to the face mask, which is then connected to the elbow, the design of the elbow connecting directly to the face mask offers improvements. This new design provides additional radial support from the elbow to the frame and a restricted fixation of the frame by the elbow via a baffle. Furthermore, the connection points have been reduced from two to one, making the connections between components sturdier and less likely to loosen. This simplifies the overall structure formed by the respiratory components, reducing the overall component failure rate. In the existing connection method, the face mask attaches directly to the patient's face through straps or other means, and the frame is connectable to the face mask through a snap-fit connection. The connection of the face mask relative to the frame is more stable and less likely to detach. However, when the elbow connects to the frame, the frame has an outward force that is opposite to the patient's face, increasing the risk of the frame detaching due to the force. By having the elbow connect directly to the face mask, this potential issue is avoided. As long as the face mask is securely attached to the patient's head using straps or other methods, the connection between the elbow, frame, and face mask will not cause the face mask to detach from the patient's face.
4. Compared to the current elbow design that connects to the frame using clamps on both sides of the elbow, the new design that connects directly to the face mask simplifies the overall structure of the elbow. Apart from the baffle, the outer wall of the elbow is essentially smooth and tidy, simplifying the complexity of the mold in manufacturing, and thereby reducing the production costs of the elbow. Moreover, without the need for specially designed and complex external connection structures, the reduction in the number of connection structures streamlines the manufacturing and assembly processes. The reduced component structure also means less demand for the corresponding materials, and there's no need for secondary materials besides the primary material of the elbow, resulting in cost savings both in terms of processing and materials. For the frames, the method of the elbow with a baffle directly connecting to the face mask eliminates the need for additional fasteners at the connection point of the frame to secure the frame. The connectors of the frame only need to be manufactured as a simple, smooth-walled opening compatible with the elbow. This represents a simplified approach for the production molds of the frame, resulting in a significant reduction in mold costs.
5. For components used in obstructive sleep apnea therapy, cleanliness is crucial. Given the frequent use, patients need to regularly clean and maintain these components. The junctions where components connect are the areas most susceptible to the accumulation of dirt and grime. If not cleaned thoroughly or regularly, residues, oils, and secretions can clog passages, fostering the growth of bacteria, viruses, and other microorganisms. This can lead to allergic reactions and skin infections. Simplifying the connection structure between the elbow and frame reduces the areas where dirt and grime can accumulate, enabling patients to clean more effectively. This not only reduces health risks but also ensures patient safety and enhances treatment compliance. Moreover, with fewer components, the carbon emissions during the manufacturing process will also be correspondingly reduced.

### BRIEF DESCRIPTION OF THE DRAWINGS

FIG. 1 is a structural schematic diagram of an elbow assembly employing a snap-fit design in accordance with an example embodiment;

FIG. 2 is a front view of an elbow assembly employing a snap-fit design in accordance with an example embodiment;

FIG. 3 is a structural schematic diagram of an elbow assembly in the form of a ball socket combined with a hinge in accordance with an example embodiment;

FIG. 4 is a structural schematic diagram of an elbow assembly in the form of a ball socket combined with a hinge and then connected to a face mask via a connector in accordance with an example embodiment;

FIG. 5 is a sectional view in the direction of A-A in FIG. 2;

FIG. 6 is a schematic diagram of a ventilation port of an elbow assembly in accordance with an example embodiment;

FIG. 7 is a diagram showing a ratio of the inner diameter and the outer diameter of exhaust holes of an elbow assembly in accordance with an example embodiment;

FIG. 8 is an illustration of the coordinated use of an elbow assembly in accordance with an example embodiment;

FIG. 9 is an exploded structural schematic diagram of an elbow assembly in use in accordance with an example embodiment;

FIG. 10 is a schematic diagram illustrating the airflow when an elbow assembly is directly connected to a face mask in accordance with an example embodiment;

FIG. 11 is a schematic diagram illustrating the airflow when an elbow is connected to a frame and the frame is further connected to a face mask;

FIG. 12 is a magnified view of a structural connection between an elbow on an elbow assembly and a face mask in accordance with an example embodiment;

FIG. 13 is a diagram illustrating the forces acting on the frame in accordance with an example embodiment;

FIGS. 14A and 14B are various forms or configurations of a baffle on an elbow assembly in accordance with an example embodiment;

FIGS. 15A, 15B and 15C are various forms or configurations of a stepped portion on the elbow component in accordance with an example embodiment;

FIGS. 16A and 16B are different forms or configurations of the connection between an elbow assembly and a face mask in accordance with an example embodiment;

FIG. 17 is a structural diagram of an elbow assembly with exhaust and noise reduction functionality;

FIG. 18 is a structural diagram of an elbow assembly demonstrating noise reduction directly achieved by a noise reduction mesh in accordance with an example embodiment;

FIG. 19 is a schematic diagram illustrating the arrangement of soft rubber at the connection point between an elbow and a face mask in accordance with an example embodiment;

FIG. 20 is a schematic diagram depicting the connection between an elbow assembly and a nasal mask in accordance with an example embodiment;

FIGS. 21A and 21B are schematic diagrams illustrating an elbow assembly with a ventilation port on an elbow or a connector in accordance with an example embodiment.

### DETAILED DESCRIPTION

In order to facilitate the understanding of the disclosure, a more comprehensive description of the disclosure will be provided with reference to the accompanying drawings. The drawings present typical embodiments of the disclosure. However, the disclosure can be implemented in many different forms and is not limited to the embodiments described herein. Instead, the purpose of providing these embodiments is to make the disclosure more thorough and comprehensive.

Unless otherwise defined, all technical and scientific terms used herein have the same meaning as commonly understood by those skilled in the technical field to which the disclosure belongs. The terms used in the description of the disclosure herein are intended to describe specific embodiments for the purpose of illustration and are not intended to limit the disclosure.

The present disclosure addresses the issue in the market where traditional elbows require the frame to be initially connected to a face mask before being connected to an elbow. This results in multiple connection points, leading to potential loosening and insecure sealing at these points. Moreover, the wearing process is cumbersome. In response to this, an elbow assembly that directly connects through the frame to the face mask is provided herein. This reduces the number of connection points from two to one, lowering the likelihood of faults in the therapy accessory assembly and insecure sealing between components. The direct connection of the elbow to the face mask eliminates the need for auxiliary connecting parts like clips, making the elbow more convenient to use and cost-effective in terms of processing. This connection method requires only a smooth central hole in the frame for the elbow to pass through, modularizing the frame and enhancing compatibility between the frame, face mask, and elbow.

The following provides a detailed illustration of several structures of the elbow assembly described herein, which directly connects to the face mask, through specific examples.

### Embodiment 1

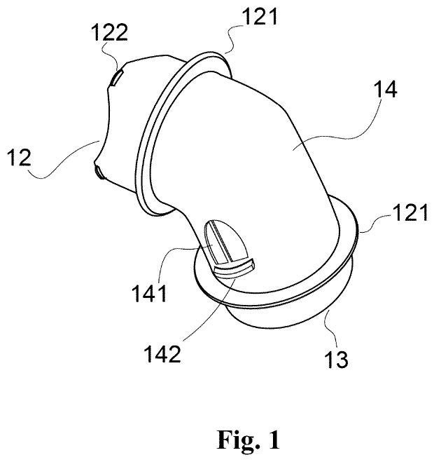

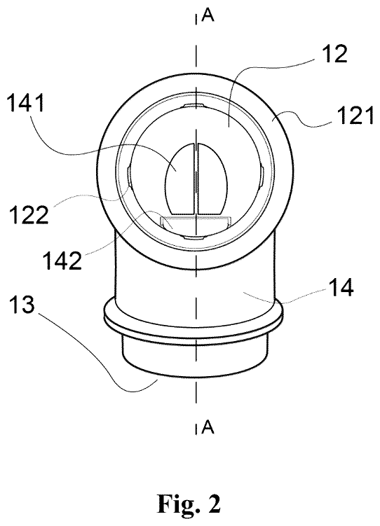

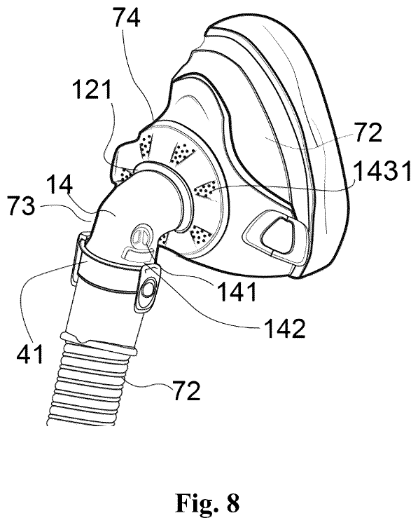

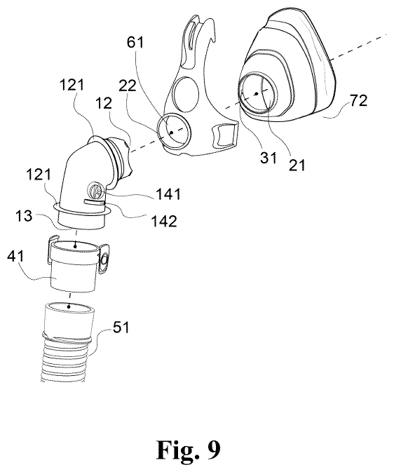

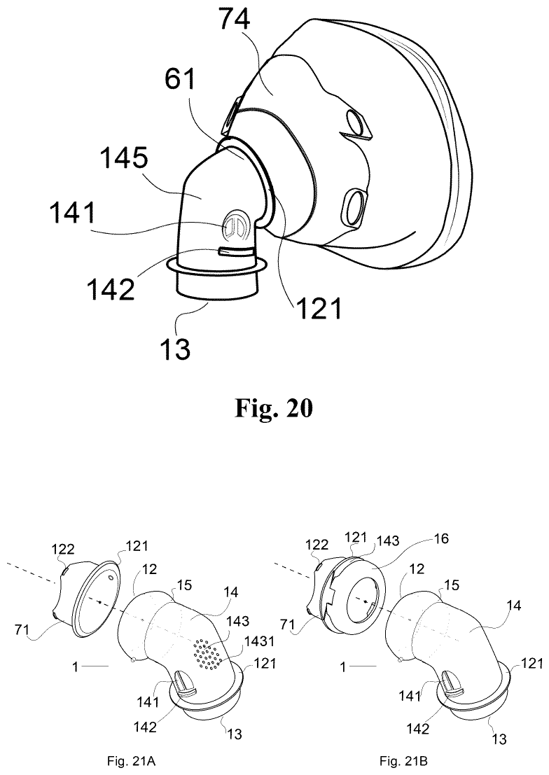

This embodiment provides an elbow assembly 1 that directly connects to the face mask 72 to deliver continuous positive airway pressure (CPAP) to improve sleep breathing disorders. The elbow assembly 1 includes an elbow 73, an anti-asphyxiation valve, and a quick-release connector 41, as referenced in FIGS. 1-2, 5, and 8-16, as well as FIG. 20. The elbow assembly illustrated in this embodiment includes a first interface 12, a second interface 13, and an annular channel 14 of the shaft bend between the first interface 12 and the second interface 13. A shaft bend is defined as a bend formed by a consistent radial force, where radial refers to a direction along the diameter or radius, or perpendicular to the axis. This annular channel 14 is configured to direct and modify the airflow direction as well as diminish the direct impact of airflow on the user. The angle formed by the intersection of the axis of the first interface 12 and the axis of the second interface 13 of the elbow 73 constitutes the angle of the elbow 73. This angle ensures that the continuous positive pressure airflow can pass smoothly through the elbow 73 and also determines the extent of the impact of the breathing tube 51 on the user. If the angle is less than 90°, it can result in airflow collisions within the elbow, leading to disruptions in flow, and the force required for removing and installing the breathing tube 51 would be directed towards the patient, which is not conducive for installation. Hence, the angle should be equal to or greater than 90°. The second interface 13 of the elbow 73 is configured to receive pressurized gas from a ventilator, and the second interface 13 of the elbow 73 can be connectable to a quick-release connector 41. The quick-release connector 41 is configured to have connection portions integrally molded or separately molded with its main body on both sides for clamping and snapping together. The quick-release connector 41 has a first end and a second end, with the first end configured to detachably connect to the second interface end of the elbow 73, and the second end configured to connect to the breathing tube 51. The quick-release connector 41 is rotatable relative to the elbow 73 after a connection to the second interface 13 of the elbow 73, enhancing the flexibility of the therapeutic accessory assembly and increasing the user's range of motion.

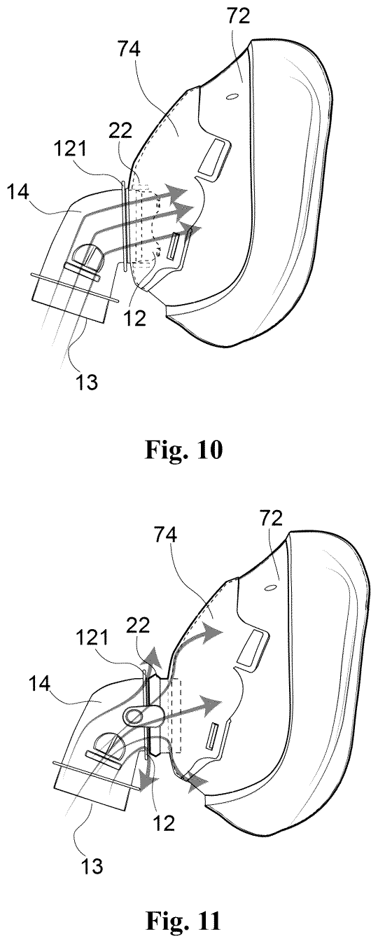

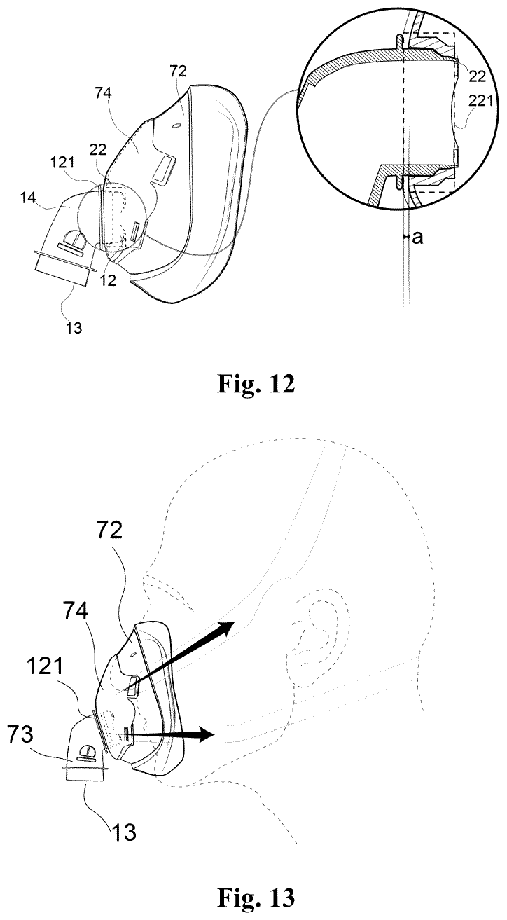

The first interface 12 of the elbow 73 is directly connectable to the face mask 72 through the central opening 21 on the face mask 72, and there can be different modes of connection between the elbow 73 and the face mask 72. In one mode of connection, the first interface end 12 of the elbow 73 is configured to include at least one stepped portion 122, which is configured to come into close contact with the inner wall of the extended interface 22 at a central opening 21 of the face mask 21 when the stepped portion 122 and the face mask 72 are connected, preventing the elbow 73 from easily detaching when a force is applied to the elbow 73 ((i.e., the force required for the detachment of the elbow 73 is less than 15N) in a direction opposite to the patient's face, and enabling mutual rotation between the elbow 73 and the face mask 72. The stepped portion 122 creates a certain gap between the first interface 12 and the extended interface 22 of the face mask 72 when the elbow 73 is engaged with the face mask 72, enabling the rotational function of the elbow 73. The stepped portion 122 also ensures a seal between the elbow 73 and the face mask 72. The stepped portion 122 at the first interface end of the elbow 73 can take various forms (as shown in FIGS. 15A, 15B, and 15C), including but not limited to a form of a protrusion (as depicted in FIGS. 15A and 15B) or recess (as depicted in FIG. 15C) on the outer wall of the first interface end of the elbow 73, which fits with a corresponding recess or protrusion on the extended interface 22 of the face mask 72. The height difference of the stepped portion 122 at the outer wall of the first interface 12 of the elbow 73 is at or between 1 to 3 mm. A height that is too excessive is detrimental for the entry of the elbow 73 into the extended interface 22 of the face mask 72, while a height that is too low fails to form a secure engagement with the extended interface 22 of the face mask 72, making it easy to inadvertently pull out the elbow 73. The connection of the first interface end of the elbow 73 with the face mask 72 occurs as the patient pushes the elbow 73 along the axis of the central opening 21 of the face mask 72 into the extended part of the face mask 72. This pushing force is converted into a radial squeezing force on the outer wall near the first interface 12 of the elbow 73 by the extended interface 22 of the face mask 72, and a radial pushing force by the elbow 73 on the extended interface 22 of the face mask 72. This results in deformation at either the first interface end of the elbow 73 or the extended interface 22 of the face mask 72, allowing the elbow 73 to engage and rotate with the face mask 72 through the stepped portion 122 at the first interface end. Once insertion is complete, the first interface end of the elbow 73 returns to its original shape, maintaining the security of the connection. When the first interface end of the elbow 73 and the extended part of the face mask 72 are subjected to the same radial force, to ensure that one of the elbow 73 and the face mask 72 undergoes greater deformation, in the case of equal elastic moduli of materials, a ratio of a uniform wall thickness at the stepped portion 122 near the first interface 12 of the elbow 73 to an area ratio between a contact area where the first interface 12 on the elbow 73 and the extended interface 22 of the face mask 72 make contact, relative to an annular area formed by their contact, is not equal to the value of the uniform wall thickness of the extended interface 22 of the face mask 72. (When the first interface 12 of the elbow 73 has a flat (i.e., complete) circle without notches, the ratio of the contact area between the first interface 12 of the elbow 73 and the extended interface 22 of the face mask 72 to the annular area 221 formed by the contact part of the first interface 12 of the elbow 73 and the extended interface 22 of the face mask 72 is 1. When the first interface 12 of the elbow 73 has a discontinous (i.e., incomplete) circular ring with notches, this ratio is not equal to 1. When there are notches at the first interface 12 of the elbow 73, the annular area 221 formed by the contact part of the first interface 12 of the elbow 73 and the extended interface 22 of the face mask 72 is equivalent to the annular area formed by the contact part of the first interface 22 of the face mask 72 and the extended interface 22 of the face mask 72 after filling in the notches. See e.g., FIG. 12.) Additionally, to ensure the security and strength of the elbow 73 when deformation of the elbow 73 is greater than the deformation of the extended interface 22 of the face mask 72 during connection, the uniform wall thickness of the stepped portion 122 at the first interface end of the elbow 73 is less than the uniform wall thickness at the annular channel 14 of the elbow 73. Apart from changing the wall thickness, the edge of the first interface 12 of the elbow 73 can also be a discontinuous circular ring with at least one notch to facilitate snapping of the first interface end to the face mask 72, as shown in FIG. 16 (FIG. 16A depicts the form with a notch, while FIG. 16B illustrates the elbow 73 being a flat circle without a notch). Furthermore, the direct connection of the first interface 12 of the elbow 73 to the extended interface 22 of the face mask 72 ensures that the airflow in the channel at the connection between the elbow 73 and the face mask 72 does not involve the frame 74. In contrast to the connection form where the frame 74 connects the face mask 72 and then the elbow 73 connects the frame 74, this configuration involving only the elbow 73 and the face mask 72 prevents airflow from leaking out of the gap at the connection between the elbow 73 and the frame 74, thereby enhancing the sealing of the connection, as shown in FIGS. 10 and 11.

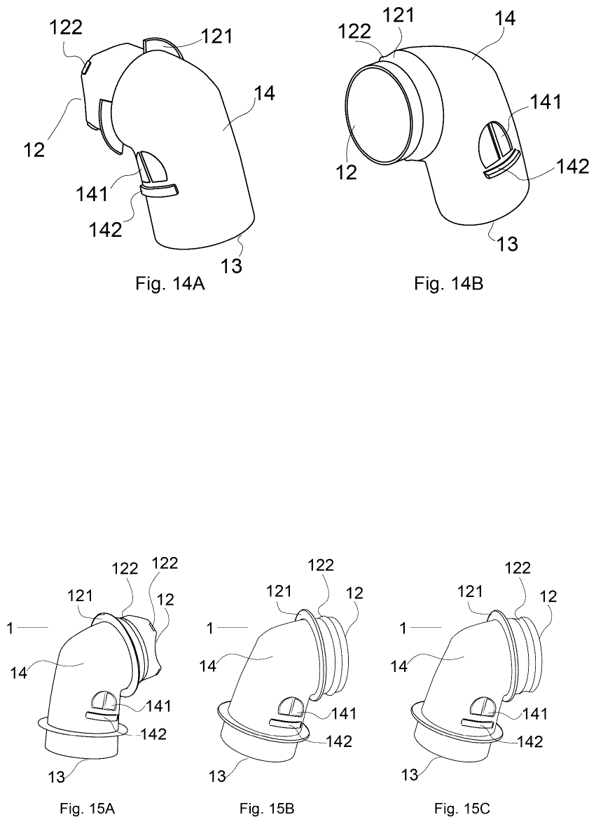

The elbow assembly 1 includes a baffle 121 near the first interface 12, which is configured to ensure that upon a connection of the elbow 73 to the face mask 72, there exists a gap between the inner surface of the baffle facing the patient's face and the outer surface of the central opening 21 on the face mask 72, with the gap being configured to at least accommodate a wall thickness of the frame 74. The gap is at least equal to the thickness at the connection point of the frame, which is 0.5 mm (as shown in gap “a” in FIG. 12). In short, the distance between the outer surface of the central opening 21 of the face mask 72 and the inner surface facing the patient's face of the baffle 121 is greater than or equal to the thickness at the connection point of the frame that accommodates the frame 74. Additionally, the distance from the first interface 12 of the elbow 73 to the inner surface of the baffle 121 facing the patient's face is greater than the distance from the outer surface of the central opening 21 of the face mask 72 to the outermost of the extended interface 22. This configuration is to limit the forward and backward movement of the frame 74 relative to the face mask 72 and the elbow 73. See e.g., FIG. 14. The baffle 121 can take the form of any arbitrary arc-shaped piece or any other shape on the circular ring or circular ring piece. When the face mask 72, frame 74, and elbow 73 are connected, the inner surface of the baffle 121 facing the patient's face comes into contact with the outer surface of the frame opening 61 that does not face the patient's face. Moreover, the distance between the inner surface of the baffle 121 facing the patient's face and the first interface 12 of the elbow 73 is at least 2 mm. Since the face mask 72, frame 74, and elbow 73 are connected along an axis, and the outer surface of the frame opening 61 is parallel to the outer surface of the central opening 21 of the face mask 72, the baffle 121 needs to be parallel to the outer surface of the frame opening 61 to effectively limit the forward and backward movement of the frame 74. Therefore, the baffle 121 is configured to be perpendicular to the axis of the interface connectable to the face mask 72. The function of the baffle 121 in limiting the forward and backward movement of the frame 74 is based on its radial height relative to the outer wall 145 of the annular channel of elbow 73. Thus, its height needs to be at least 2 mm to effectively limit the forward and backward movement of the frame 74. To withstand potential damage, the baffle 121 has a certain thickness, which is at or between 0.5 mm to 8 mm. The baffle 121 can also be a wall of the elbow 73 formed relative to the stepped portion 122 at the first interface 12. In other words, the baffle has at least two forms: one is a radially protruding part on an outer wall of the elbow 73 (as shown in FIG. 14A), and the other is formed by the stepped portion 122 on the elbow 73 (as depicted in FIG. 14B).

The elbow assembly 1 further includes an anti-asphyxiation valve, including an anti-asphyxiation valve opening 141 and a silicone piece 142 at the lower end of the anti-asphyxiation valve opening 141. The anti-asphyxiation valve opening 141 is provided on the wall of the annular channel on the elbow 73, configured to allow breathing via the anti-asphyxiation valve opening 141 in an absence of continuous positive pressure in the elbow 73, preventing the patient from asphyxiation. The anti-asphyxiation valve opening 141 can include one or more openings. The silicone piece 142 is configured to block the anti-asphyxiation valve opening 141 when the ventilator is in operation, and to open the anti-asphyxiation valve opening 141 when the ventilator is not in operation. The area of a side of the silicone piece 142 close to the anti-asphyxiation valve is greater than a total area of the anti-asphyxiation valve opening 141 to prevent the silicone piece 142 from being blown out. The silicone piece 142 can be disassembled and installed from the elbow 73 by deformation and at least a portion of it is in contact with the external environment. To ensure smooth airflow through the anti-asphyxiation valve, the anti-asphyxiation valve forms a symmetry with the silicone piece at the lower end of the valve at an axis of the second interface end of the elbow 73.

In some other implementations, the elbow assembly 1 can also connect to a nasal mask (as shown in FIG. 20).

### Embodiment 2

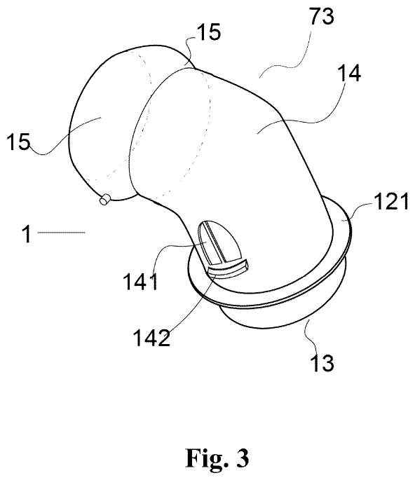

This embodiment provides an elbow assembly 1 directly connectable to the face mask 72 for delivering continuous positive pressure gas to the patient's airway to improve sleep breathing disorders. The elbow assembly 1 includes an elbow 73 and an anti-asphyxiation valve. The anti-asphyxiation valve includes an anti-asphyxiation valve opening 141 and a silicone piece 142 at the lower end of the anti-asphyxiation valve opening 141. See e.g., FIG. 3. FIG. 3 provides a structural schematic diagram of the elbow assembly 1 in this embodiment. In the embodiment of the disclosure discussed herein (as shown in FIG. 3), the difference from the elbow assembly 1 in embodiment one is as follows: a partially spherical-shaped protrusion 15 is provided at the first interface end of the elbow, and the outer wall near the first interface end has two protrusions facing outward or two grooves facing inward. When the elbow 73 is connected to the face mask 72, the protrusions or grooves adapt with a receiver 31 on the inner wall of the central opening 21 on the face mask 72 to form a hinged connection to allow the elbow 73 to be rotated relative to the face mask 72 along the axis of the formed hinge, and the partially spherical-shaped protrusion 15 at the first interface end 12 is configured to ensure smooth rotation of the elbow 73 and to prevent air leakage during the rotation of the elbow 73. The two protrusions facing outward or two grooves facing inward are configured to achieve rotation within a certain angle range in the vertical direction.

In another implementation, the elbow assembly 1 includes an elbow 73, an anti-asphyxiation valve, and a quick-release connector 41. The anti-asphyxiation valve includes an anti-asphyxiation valve opening 141 and a silicone piece 142 positioned at the lower end of the anti-asphyxiation valve opening 141. In this connection configuration, the elbow 73 achieves a rotation connection with the face mask 72 at a certain angle rather than a full 360° rotation. The quick-release connector 41, in its connection to the second interface 13 of the elbow 73, allows the tube to rotate 360° relative to the elbow 73. This further enhances the range of movement for the patient.

### Embodiment 3

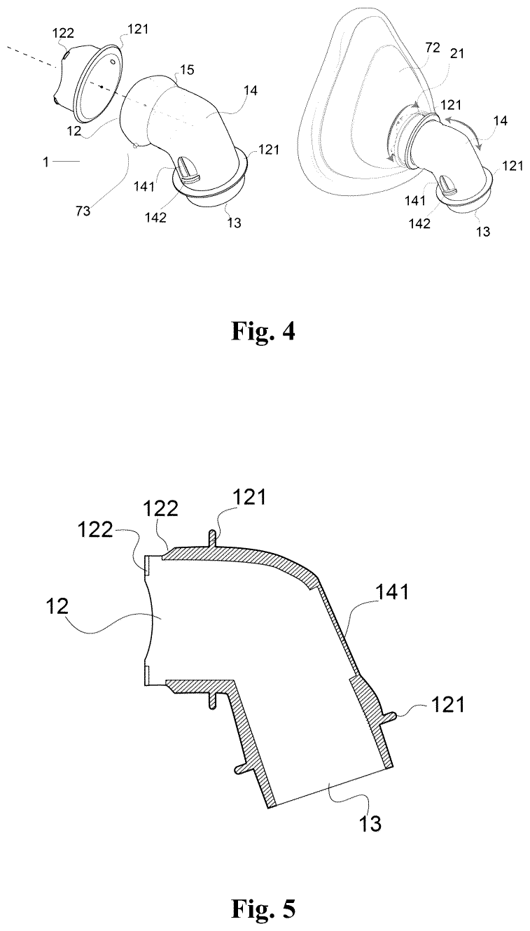

This embodiment provides an elbow assembly 1 directly connecting to the face mask 72, configured to provide continuous positive air pressure to the patient's airway and to ameliorate sleep-related breathing disorders. This elbow assembly 1 includes an elbow 73, an anti-asphyxiation valve, and a connector 71. The anti-asphyxiation valve includes an anti-asphyxiation valve opening 141 and a silicone piece 142 at the lower end of the anti-asphyxiation valve opening 141, as shown in FIG. 4. This embodiment presents a structural schematic diagram of the elbow assembly 1. This embodiment depicted in FIG. 4 is different from Embodiment 1 in that the elbow assembly 1 includes a connector 71 directly connecting to the face mask 72. The connector 71 is provided with a first end connectable to the elbow 73 and a second end directly connectable to the face mask 72. The second end of the connector 71 includes at least one stepped portion 122, which is configured to come into close contact with the extended interface 22 at the central opening 21 of the face mask 72 when the stepped portion 122 and the face mask 72 are connected, preventing the connector 71 from easily detaching (i.e., the force required for the detachment of the elbow 73 is less than 15N) when a force is applied to the connector 71 in a direction opposite to the patient's face. The first end of the connector 71 connectable to the elbow 73 is connected to the elbow 73 through a snap-fitting, ball-and-socket joint, or a combination of hinge and ball-and-socket joint. The first end of the connector 71 could be connected to the elbow 73 detachably or non-detachably. Additionally, the material of the connector 71 can be identical or different from that of the elbow 73. The elbow 73 has a protrusion at the first interface end that is partially spherical-shape d, and the partially spherical-shaped protrusion 15 is provided with a hinge structure that allows for relative rotation. The partially spherical-shaped protrusion 15 of the elbow 73 is connectable to the connector 71. The structural configuration at the first interface end of the elbow 73 enables the rotation of the elbow 73 relative to the face mask 72 along the axis of the hinge. Through the spherical protrusion 15 and the snap-fitting, a gas-tight seal is formed, securing the elbow 73. The connector 71 provided at the first interface end of the elbow 73 enables arbitrary rotation of the elbow 73 along the axis of the first interface end relative to the face mask 72. In this connection configuration, the elbow assembly 1, through the connector 71, not only achieves the function of arbitrary rotation along the axis of the first interface end but also incorporates the functionality of rotation along the axis of the hinge at a certain angle. This enhanced flexibility in the connection of the elbow assembly 1 reduces the impact of patient movement during wearing, enhancing overall comfort by minimizing the influence of patient movement on the entire assembly, making it more flexible than those provided in Embodiment 1 and Embodiment 2. In some implementations, the connector 41 is not integrally formed with the elbow 73.

### Embodiment 4

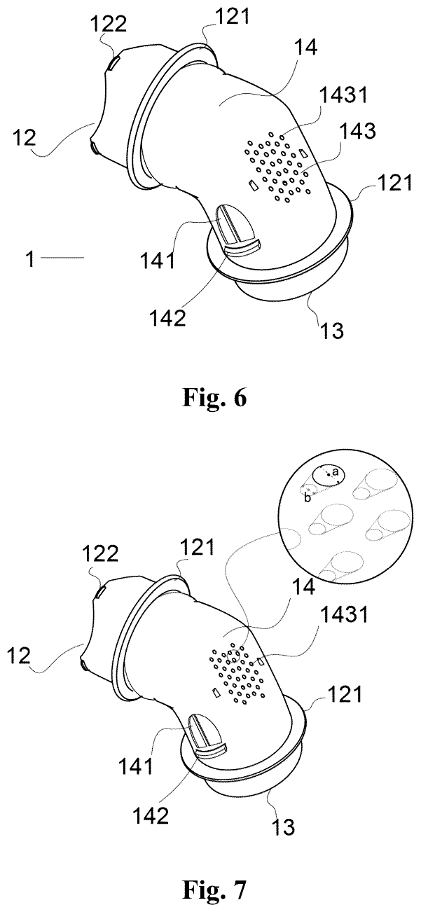

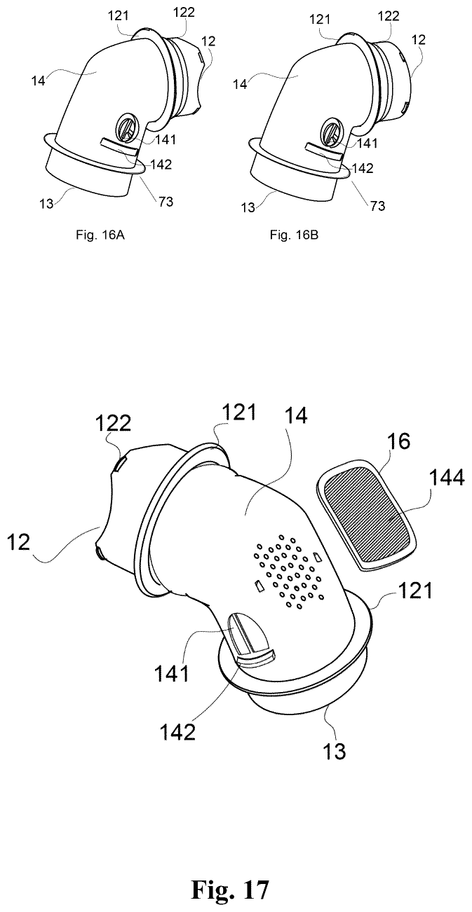

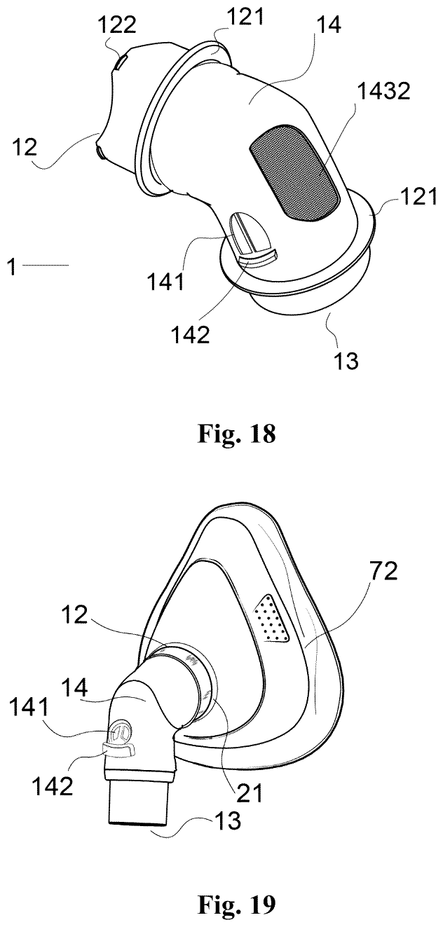

This embodiment provides an elbow assembly 1 that connects directly to a face mask 72. The assembly is used to supply continuous positive airway pressure to a patient's airway, improving sleep breathing disorders. The elbow assembly 1 includes an elbow 73, a connector 71, an anti-asphyxiation valve, and a ventilation port 143. The anti-asphyxiation valve includes an anti-asphyxiation valve opening 141 and a silicone piece 142 provided at its lower end, as shown in FIGS. 6-7, 17-18, and 21. The embodiment offers a structural schematic diagram of the elbow assembly 1. In the embodiment shown in FIGS. 6-7, 17-18, and 21, the difference from Embodiment 1 of the elbow assembly 1 is the inclusion of a ventilation port 143. This port 143, having at least one ventilation hole, allows the patient's exhaled waste gases to flow to the external world. The ventilation port 143 can be provided on the connector 71, as shown in FIG. 21B, or on the elbow 73, as illustrated in FIG. 21A. The ventilation port 143 may include a noise reduction feature (set up with a noise reduction device). The ventilation port 143 includes ventilation holes 1431 with noise-reducing material 144 or is made directly from a noise-reducing mesh 1432, as shown in FIG. 18, used to disperse the patient's exhaled waste gases. The ventilation holes 1431 can be configured to form a certain taper between the outer aperture, which is in contact with the air, and its corresponding inner aperture, as seen in FIG. 7. Alternatively, the inner aperture and outer aperture are of equal size. The shape of the ventilation port 143 and the ventilation hole 1431 can be O-shaped, square, or any other shape. To cover the noise-reducing material 144 over the ventilation holes 1431, the elbow 73 can have at least one port cover 16, as shown in FIG. 17. The port cover 16 is used for connecting to an outer wall of the annular channel in contact with air to secure the noise-reducing material 144. The port cover 16 can be made of other materials that are not integrally formed with the elbow 73, or it can be composed of the same material as the elbow 73. Alternatively, it can be formed integrally with the connector 71.

The noise-reducing material 144 includes, but is not limited to, noise-reducing cotton or noise-reducing mesh 1432. Fiber-based sound-absorbing materials or foam-based sound-absorbing materials can be used, which can be made from fabrics, nylon, polypropylene, or other materials. The way in which the elbow 73 is fixed with the noise-reducing material 144 can be through injection molding, ultrasonic bonding, heat compression, or other methods. It can also involve adhesion using adhesives such as glue, tape, etc., or via fasteners like snap-fits, knobs, or clamps. Additionally, it can be assisted by a third component like the port cover 16, which is configured to secure the noise-reducing material 144 on the outer surface directly in contact with the air of connector 71. The noise-reducing material 144 is appropriately sized to fit between the ventilation port 143 of the elbow 73 and the port cover of the elbow 73. The width of the noise-reducing material is smaller than that of the port cover. It is attached to the outer surface of the ventilation port 143 of the elbow 73 to allow the dispersion of exhaled gases into the external environment. The maximum thickness of the noise-reducing material 144 is 9 mm, and it weighs a maximum of 7 g. The addition of the ventilation port 143 and noise-reducing function expands the options for patients in selecting compatible face masks 72 and frames 74. Patients now have the flexibility to choose face masks 72 without ventilation ports to use with elbows 73 that have ventilation ports 143, or to use face masks 72 without ventilation ports 143 with elbows 73 lacking ventilation ports 143 and connectors that have ventilation ports 143. This widens the range of choices for users.

### Embodiment 5

This embodiment provides an elbow assembly 1 directly connectable to the face mask 72 for delivering continuous positive pressure gas to a patient's airway to improve sleep breathing disorders. The elbow assembly 1 includes an elbow 73, an anti-asphyxiation valve, and a quick-release connector 41, with the anti-asphyxiation valve including an anti-asphyxiation valve opening 141 and a silicone piece 142 provided at the lower end of the anti-asphyxiation valve opening 141, as shown in FIG. 19. A structural schematic diagram of the elbow assembly 1 is provided in this embodiment. In the illustrated embodiment of the disclosure discussed herein, as illustrated in FIG. 19, a difference from the elbow component 1 in Embodiment 1 is that the first interface end of the elbow 73 is made of soft rubber. It is configured as a ring of soft rubber fixed to the opening of the first interface 12 of the elbow 73, extending inwardly towards the patient's face or outwardly opposite to it. When the elbow 73 is connected to the face mask 72, there is no stepped portion 122 set at the first interface 12 of the elbow 73. It relies solely on the frictional force between the soft rubber at the first interface 12 of the elbow 73 and the extended interface 22 of the face mask 72 for fixation. This simplifies the structure of the elbow 73, reduces production costs, and, to some extent, modularizes the elbow 73. Because the soft rubber has a certain elasticity, it accommodates the face mask 72 with the extended interface 22 having apertures that do not differ significantly from each other, and weakens the selective impact of the structure at the extended interface 22 of the face mask 72 on choosing the elbow 73. Patients only need to choose an elbow 73 with a soft rubber circumference within a certain range that matches the face mask 72. There is no need to consider the corresponding structure at the interface, and the soft rubber can also be provided at the extended interface 22 of the face mask 72 for connection with the elbow 73.

Furthermore, the technical features in the various embodiments mentioned above can be combined as needed to obtain an elbow assembly directly connected to the face mask, incorporating all or some of the aforementioned technical features.

Implementing the elbow assembly for direct connection to a face mask of this disclosure has at least the following beneficial effects:
1. By having the elbow directly connect with the face mask without a frame, it reduces the number of seams at the connection points between components. The continuous positive pressure airflow from the ventilator passes through the elbow and the patient interface via a breathing tube, and enters the patient's airway. During this process, there is a potential for airflow to leak from the seams. Reducing the number of seams decreases the risk of airflow leakage, thereby improving the overall sealing of the respiratory treatment component system. Traditional installation methods required alignment at two separate locations, first aligning the frame with the fixed face mask and then aligning the elbow with the fixed frame. This disclosure simplifies the installation process from three steps to two. It is only necessary to place the frame onto the elbow before connecting the elbow to the face mask. This optimization of the installation process not only reduces the error rate and saves time but also enhances the user experience, making it easier for people with limited dexterity to operate.
2. In this disclosure, during the installation and disassembly process, the frame only passes through the first interface of the elbow. The frame is secured to the face mask and the elbow through the shape of the face mask and the baffle of the elbow. Therefore, the most critical and indispensable structure of the frame at the connection is the frame opening that passes through the first interface end of the elbow. This disclosure simplifies the connection of the frame to a smooth-walled frame opening, modularizing the frame. For different patient needs, as long as the frames have the same-sized opening that can pass through the first interface end of the elbow; they can be securely connected to the elbow and face mask. This eliminates the need for designing frames with different connecting ports to match various face masks and elbow interfaces. Similarly, there's no need to design face masks and elbows with different connecting ports to fit the frames. The reduction in processing steps for connecting ports significantly reduces the complexity and R&D costs for the elbow; face mask, and frame. It also simplifies the market choices for frames. Additionally, the reduction in structure and processing steps leads to less wastage of raw materials. This is a crucial step towards carbon neutrality, further aligning with the goals of alleviating the environmental and climate change burden, promoting sustainable development, and achieving carbon neutrality.
3. Compared to the connection style where the frame connects to the face mask, which is then connected to the elbow; the design of the elbow connecting directly to the face mask offers improvements. This new design provides additional radial support from the elbow to the frame and a restricted fixation of the frame by the elbow via a baffle. Furthermore, the connection points have been reduced from two to one, making the connections between components sturdier and less likely to loosen. This simplifies the overall structure formed by the respiratory components, reducing the overall component failure rate. In the existing connection method, the face mask attaches directly to the patient's face through straps or other means, and the frame is connectable to the face mask through a snap-fit connection. The connection of the face mask relative to the frame is more stable and less likely to detach. However, when the elbow connects to the frame, the frame has an outward force against the patient's face, increasing the risk of the frame detaching due to the force. By having the elbow connect directly to the face mask, this potential issue is avoided. As long as the face mask is securely attached to the patient's head using straps or other methods, the connection between the elbow, frame, and face mask will not cause the face mask to detach from the patient's face.
4. Compared to the current elbow design that connects to the frame using clamps on both sides of the elbow; the new design that connects directly to the face mask simplifies the overall structure of the elbow. Apart from the baffle, the outer wall of the elbow is essentially smooth and tidy, simplifying the complexity of the mold in manufacturing, and thereby reducing the production costs of the elbow. Moreover, without the need for specially designed and complex external connection structures, the reduction in the number of connection structures streamlines the manufacturing and assembly processes. The reduced component structure also means less demand for the corresponding materials, and there's no need for secondary materials besides the primary material of the elbow, resulting in cost savings both in terms of processing and materials. For the frames, the method of the elbow with a baffle directly connecting to the face mask eliminates the need for additional fasteners at the connection point of the frame to secure the frame. The connectors of the frame only need to be manufactured as a simple, smooth-walled opening compatible with the elbow. This represents a simplified approach for the production molds of the frame, resulting in a significant reduction in mold costs.
5. For components used in obstructive sleep apnea therapy, cleanliness is crucial. Given the frequent use, patients need to regularly clean and maintain these components. The junctions where components connect are the areas most susceptible to the accumulation of dirt and grime. If not cleaned thoroughly or regularly, residues, oils, and secretions can clog passages, fostering the growth of bacteria, viruses, and other microorganisms. This can lead to allergic reactions and skin infections. Simplifying the connection structure between the elbow and frame reduces the areas where dirt and grime can accumulate, enabling patients to clean more effectively. This not only reduces health risks but also ensures patient safety and enhances treatment compliance. Moreover, with fewer components, the carbon emissions during the manufacturing process will also be correspondingly reduced.

The various technical features of the embodiments described above can be combined in any way. To keep the description concise, not all possible combinations of the technical features in the above embodiments have been described. However, as long as these combinations of technical features are not contradictory, they should be considered within the scope documented in this specification. It must be noted that as used herein and in the appended claims, the singular forms “a”, “an”, and “the” include their plural equivalents, unless the context clearly dictates otherwise.

The embodiments described above only represent several implementation methods of the disclosure. While the descriptions are specific and detailed, they should not be understood as limiting the scope of the patent. It should be noted that ordinary skilled artisans in this field can make various modifications and improvements without departing from the conceptual framework of the disclosure. All such modifications and improvements are within the scope of protection of the present disclosure. Therefore, the scope of protection of the patent for this disclosure is determined by the appended claims.

1. A facemask assembly, configured to deliver continuous positive pressure gas to a patient's airway to ameliorate sleep-associated breathing disorders, the facemask assembly comprising:
a face mask;
a frame on an exterior surface of the face mask; and
an elbow, having a first interface for direct connection to the face mask, a second interface to receive pressurized gas, and an annular channel with a shaft bend between the first interface and the second interface, wherein an angle between an axis of the first interface and an axis of the second interface of the elbow is greater than 90 degrees;
wherein a first interface end on the elbow includes at least one stepped portion, configured to come into contact with an inner wall of an extended interface at a central opening of the face mask when the stepped portion and the face mask are connected to allow mutual rotation between the elbow and the face mask;
wherein the elbow has a baffle outwardly extending from an outer surface of the elbow near the first interface, configured to ensure that upon a connection of the elbow with the face mask, there exists a gap between an inner surface of the baffle facing the patient's face and an outer surface of the face mask, wherein the frame is retained in the gap by the gap accommodating a wall thickness of the frame to limit forward and backward movement of the frame relative to the face mask and the elbow;
wherein the elbow has a second baffle near the second interface, the second baffle extending continuously around the outer surface of the elbow;
wherein the facemask assembly further includes: an anti-asphyxiation valve, which includes an anti-asphyxiation valve opening and a silicone piece positioned at a lower end of the anti-asphyxiation valve opening, wherein the anti-asphyxiation valve opening is provided on a wall of the annular channel on the elbow, configured to allow breathing via the anti-asphyxiation valve opening in an absence of continuous positive pressure in the elbow, to prevent the patient from asphyxiation; and
wherein a distance between the inner surface of the baffle at the first interface end of the elbow facing the patient's face, and the first interface of the elbow, is no less than 2 mm.

2. The facemask assembly according to claim 1, wherein an edge of the first interface is a discontinuous circular ring, the ring including at least one notch to facilitate a snap-fitting of the first interface end with the face mask.

3. The facemask assembly according to claim 1, wherein the baffle is a radially protruding part on an outer wall of the elbow or is formed by the stepped portion on the elbow.

4. The facemask assembly according to claim 1, wherein the anti-asphyxiation valve opening forms a symmetry with the silicone piece at the axis of a second interface end of the elbow.

5. The facemask assembly according to claim 1, wherein the anti-asphyxiation valve opening is configured to have one or more openings.

6-16. (canceled)

17. A facemask assembly, configured to deliver continuous positive pressure gas to a patient's airway to ameliorate sleep-associated breathing disorders, the facemask assembly comprising:
a face mask;
a frame on an exterior surface of the face mask; and
an elbow, having a first interface for direct connection to the face mask, a second interface to receive pressurized gas, and an annular channel with a bend between the first interface and the second interface;
a first interface end on the elbow configured to be in contact with an extended interface at a central opening of the face mask, when connected to the face mask;
the elbow having a first baffle outwardly extending from an outer surface of the elbow near the first interface, which is configured to ensure that upon a connection of the elbow to the face mask, there exists a gap between an inner surface of the first baffle facing the patient's face and an outer surface of the face mask, wherein the frame is retained in the gap by the gap accommodating a wall thickness of the frame to limit forward and backward movement of the frame relative to the face mask and the elbow, and wherein the elbow has a second baffle near the second interface, the second baffle extending continuously around the outer surface of the elbow; and
a quick-release connector, having a first end and a second end, the first end configured to be detachably connectable to a second interface end of the elbow, and the second end being configured to be connectable to a breathing tube;
wherein the facemask assembly further includes: an anti-asphyxiation valve that includes an anti-asphyxiation valve opening and a silicone piece at a lower end of the anti-asphyxiation valve opening, and wherein the anti-asphyxiation valve opening is provided on a wall of the annular channel on the elbow to allow breathing via the anti-asphyxiation valve opening in an absence of continuous positive pressure in the elbow, to prevent the patient from asphyxiation.

18. The facemask assembly according to claim 17, wherein the quick-release connector is configured to have connection portions integrally molded with its main body on two opposite sides to clamp and snap to the second interface end of the elbow.

19. (canceled)

20. The facemask assembly according to claim 17, wherein an edge of the first interface is a discontinuous circular ring, the ring including at least one notch to facilitate a snap-fitting of the first interface end with the face mask.

21. The facemask assembly according to claim 17, wherein the first interface end on the elbow includes at least one stepped portion, and the first baffle is a wall of the elbow formed relative to the stepped portion at the first interface; and wherein
the first baffle has at least one of the following characteristics:
A. being perpendicular to an axis of an interface that connects the face mask and the elbow;
B. when connected to the face mask, a distance from the first baffle to the outer surface of the central opening on the face mask being at least equal to the wall thickness of the frame at the connection point;
C. having a height of at least 2 mm;
D. having a thickness between 0.5 mm to 8 mm.

22. A facemask assembly, configured to deliver continuous positive pressure gas to a patient's airway to ameliorate sleep-associated breathing disorders, the facemask assembly comprising:
a face mask;
a frame on an exterior surface of the face mask; and
an elbow, having a first interface for direct connection to the face mask, a second interface to receive pressurized gas, and an annular channel with a shaft bend between the first interface and the second interface,
wherein a first interface end on the elbow includes at least one stepped portion, configured to come into contact with an inner wall of an extended interface at a central opening of the face mask when the stepped portion and the face mask are connected,
wherein the elbow has a first baffle outwardly extending from an outer surface of the elbow near the first interface, the frame retained in a gap between the first baffle and face mask to limit forward and backward movement of the frame relative to the face mask and the elbow,
wherein the elbow has a second baffle near the second interface, the second baffle extending continuously around the outer surface of the elbow, and
wherein the facemask assembly further includes: an anti-asphyxiation valve, which includes an anti-asphyxiation valve opening and a silicone piece positioned at a lower end of the anti-asphyxiation valve opening, and wherein the anti-asphyxiation valve opening is provided on a wall of the annular channel on the elbow, configured to allow breathing via the anti-asphyxiation valve opening in an absence of continuous positive pressure in the elbow, to prevent the patient from asphyxiation.

23. The facemask assembly according to claim 22, wherein a height of the stepped portion is between 1 mm to 3 mm.

24. The facemask assembly according to claim 22, wherein a distance between an inner surface of the first baffle, and the first interface of the elbow, is no less than 2 mm.

25. The facemask assembly according to claim 22, wherein a height of the first baffle is at least 2 mm.

26. A facemask assembly, configured to deliver continuous positive pressure gas to a patient's airway to ameliorate sleep-associated breathing disorders, the facemask assembly comprising:
a face mask;
a frame on an exterior surface of the face mask; and
an elbow, having a first interface for direct connection to the face mask, a second interface to receive pressurized gas, and an annular channel with a bend between the first interface and the second interface;
a first interface end on the elbow configured to be in contact with an extended interface at a central opening of the face mask, when connected to the face mask;
the elbow having a first baffle outwardly extending from an outer surface of the elbow near the first interface, which is configured to ensure that upon a connection of the elbow to the face mask, there exists a gap between an inner surface of the first baffle facing the patient's face and an outer surface of the face mask, wherein the frame is retained in the gap by the gap accommodating a wall thickness of the frame to limit forward and backward movement of the frame relative to the face mask and the elbow, and wherein the elbow has a second baffle near the second interface, the second baffle extending continuously around the outer surface of the elbow; and
a quick-release connector, having a first end and a second end, the first end configured to be detachably connectable to a second interface end of the elbow, and the second end being configured to be connectable to a breathing tube;
wherein the quick-release connector is configured to have connection portions on two opposite sides to clamp and snap,
wherein the facemask assembly further includes: an anti-asphyxiation valve that includes an anti-asphyxiation valve opening and a silicone piece at a lower end of the anti-asphyxiation valve opening.

27. The facemask assembly according to claim 26, wherein the quick-release connector is configured to be integrally molded with its connection portions.

28. The facemask assembly according to claim 26, wherein the anti-asphyxiation valve opening forms a symmetry with the silicone piece at an axis of the second interface end of the elbow.

29. The facemask assembly according to claim 26, wherein the anti-asphyxiation valve opening is provided on a wall of the annular channel on the elbow.

30. The facemask assembly according to claim 17, wherein the quick-release connector engages a top surface of the second baffle.

31. The facemask assembly according to claim 26, wherein the quick-release connector engages a top surface of the second baffle.
